(19) United States
(12) Patent Application Publication
(10) Pub. No.: US 2004/0166170 A1
(43) Pub. Date: Aug. 26, 2004

(54) Method for therapeutically treating mammals

(76) Inventors: Gary L. Hunter, Paulden, AZ (US); Lisa J. Hunter, Paulden, AZ (US)

Correspondence Address: WEISS & MOY PC, 4204 NORTH BROWN AVENUE, SCOTTSDALE, AZ, 85251, US

(21) Appl. No.: 10/764,944

(22) Filed: Jan. 26, 2004

Related U.S. Application Data
(60) Provisional application No. 60/450,377, filed on Feb. 26, 2003.

(51) Int. Cl. A61K 35/54; A61K 35/50

(52) U.S. Cl. 424/582; 424/583

(57) ABSTRACT

A topical application of omentum, preferably in a cream form, that takes advantage of the healing properties of omentum for the treatment of topical maladies. An internal application of omentum for the treatment of internal maladies, such as ulcers. And a method of processing the mammalian omentum for topical or internal application.

### CROSS-REFERENCE TO RELATED APPLICATIONS

This application claims priority to a corresponding provisional application U.S. Serial No. 60/450,377, filed Feb. 26, 2003 in the names of the applicants of this application.

### FIELD OF THE INVENTION

This invention relates generally to methods of therapeutically treating mammals and, more specifically, to a method for therapeutically treating mammals by topically or internally applying a therapeutically effective amount of omentum to a needed area of a mammal.

### BACKGROUND OF THE INVENTION

Omentum is a large nutrient-rich sheet of fat that is attached to the stomach and transverse colon of a mammal. The greater omentum hangs down over the intestines in the abdominal cavity while the lesser omentum is attached to the top edge of the stomach and stretches to the underside of the liver.

Omentum has been found to possess many regenerative properties. Omentum is the remnant of the membrane and yolk sac that enclosed the fetus in the womb. Omental material contains angiogenic factors that stimulate the development of blood vessels and increases blood flow, which aids growth and healing. The omentum also has neurotrophic factors which seem to nurture nerve tissue (regeneration). Scientific research into omental grafts and Omentum Transposition Surgery has revealed beneficial results for spinal cord patients, whereby angiogenic activity of the omental material increases cerebral blood flow which in turn facilitates spinal cord and brain injury repair and improves brain and neurologic function. Similarly, stroke patients undergoing Omentum Transposition Surgery were found to regain brain function, significantly improving motor, speech, and language abilities. Further research into the effects of applying omentum directly to the brain in the treatment of Alzheimer's indicates positive effects. In most medical research into the benefits of omentum, omental material has been grafted to, or applied to, an internal area of the body.

A need therefore existed for a topical application of omentum, preferably in a cream form, which takes advantage of the healing properties of omentum for the treatment of topical maladies. Omentum, and the lipids derived therefrom, can be beneficially applied to cuts, scrapes, bruises, sores, burns, sunburns, chemical burns, abrasions, acne, dry and cracked skin, chapped lips, rashes, eczema, dandruff, calluses, karatosis, seborrhea, stretch marks, wrinkle reduction, minor and major wounds, cold sores, canker sores, herpes, vaginal infections, hemorrhoids, and the nipples of nursing mothers. Omental cream is also capable of generating clitoral and penile arousal and response, as well as acting as a lubricant.

Another need existed for a non-surgical internal application of omentum for the treatment of internal maladies.

And a further need existed for a method of processing the mammalian omentum for topical or internal application.

### SUMMARY OF THE INVENTION

It is an object of the present invention to provide a topical application of omentum preferably in a cream form, that takes advantage of the healing properties of omentum for the treatment of topical maladies.

It is another object of the present invention to provide a non-surgical internal application of omentum for the treatment of internal maladies.

It is another object of the present invention to provide a method of processing the mammalian omentum for topical or internal application.

### BRIEF DESCRIPTION OF THE PREFERRED EMBODIMENTS

In accordance with one embodiment of the present invention, a method of treating mammals for topical maladies is disclosed. The method comprises the step of topically applying to a needed area on the mammal a therapeutically effective amount of a composition comprising a therapeutically effective amount of mammalian omentum.

In accordance with another embodiment of the present invention, a method of processing mammalian omentum is disclosed. The method comprises the steps of collecting raw mammalian omental material, heating the raw mammalian omental material into a liquid substance, and then filtering, tempering, and pressing the liquid substance into a finished cream form of the omental composition.

In accordance with still another embodiment of the present invention, a method of treating mammals for internal maladies is described. The method comprises the step of ingesting a therapeutically effective amount of a composition comprising a therapeutically effective amount of mammalian omentum.

In accordance with yet another embodiment of the present invention, another method of processing mammalian omentum is disclosed. The method comprises the steps of collecting raw mammalian omental material and blending the omental material into a mixture of raw lipid oil.

In accordance with another embodiment of the present invention, another method of processing mammalian omentum is disclosed. The method comprises the steps of collecting raw mammalian omental material, blending a first portion of the raw omental material into a mixture of raw lipid oil, heating a second portion of the raw omental material into a liquid substance, and combining the raw lipid oil to the liquid substance as the liquid substance cools.

The foregoing and other objects, features, and advantages of the invention will be apparent from the following, more particular, description of the preferred embodiments of the invention.

### DETAILED DESCRIPTION

Mammalian omentum is known for its medicinal value, particularly its ability to stimulate and enhance blood vessel and tissue growth. The inventor has devised a topical application of omentum, preferably in a cream form, that takes advantage of the healing properties of omentum for the treatment of topical maladies. More specifically, the inventor has created a composition that is approximately 95% mammalian omentum and also contains a therapeutically effective amount of mammalian placenta. It is preferred that bovine fetal mammalian omentum be used for the composition, however it should be understood by those skilled in the art that substantial benefit may also be obtained from using fetal omentum of other mammals. It should also be understood by those skilled in the art that substantial benefit may also be obtained from using a composition that does not contain mammalian placenta.

The immense healing potential of mammalian omentum provides a vast range for its therapeutic use. The mammalian omentum composition may be used to treat maladies and conditions that are present or manifested on the surface of the skin, such as cuts, scrapes, bruises, sores, burns, abrasions, dry skin, cracked skin, chapped lips, calluses, stretch marks, wrinkles, herpes, vaginal infections, hemorrhoids, rashes, karatosis, seborrhea, and dandruff. The composition may also be applied to an eye area and a tooth. And the composition may be applied to an internal area of the body for stimulating blood vessel and tissue growth in tendons, cartilage, nerves, and ligaments. Ingestion of the composition may also be helpful in treating internal maladies, such as ulcers. Furthermore, the composition may be used as a lubricant and also for clitoral and penile arousal.

For treatment of maladies or conditions that are present or manifested on the surface of the skin, a first embodiment of the present invention discloses a method of topically applying a therapeutically effective amount of the composition to a needed area on the mammal. The inventor has developed a cream form for the omental composition, however it should be clearly understood that many products currently topically applied to a body may also be used as vehicles for delivering the omental material. Substantial benefit may be derived from using, among other products, ointment, aftershave, soap, eye cream, skin cream, vaginal cream, deodorant, hair gel, skin filler, shampoo, sun block, or lip balm, wherein each of these products preferably contained a therapeutically effective amount of the omental material.

Certain herbs, oils, fruits, vegetables, nuts, flowers, plants, trees, roots, leaves, bark, petals, wheat germ, vitamins, minerals, animal fats, ground bone meal/marrow, and water may be added to the composition as the curative properties of each will enhance the medicinal value of the composition as a whole. Vitamin E, a natural preservative, may be added, as well as coconut extract, to provide a pleasant scent to the composition.

For treatment of maladies or conditions that are present inside a mammal's body, such as an ulcer, a second embodiment of the present invention is a method of ingesting a therapeutically effective amount of a composition comprising a therapeutically effective amount of mammalian omentum. The composition to be ingested may be any one of the compositions described herein. Although the preferred embodiment of the invention for treating an internal malady is to ingest the omental composition, it should also be understood that substantial benefit may be derived from injecting a therapeutically effective amount of the composition into the needed area.

In order to take advantage of the healing properties of mammalian omentum, the inventor has also created a method of processing a topical application of omentum, preferably in a cream form, for the treatment of topical maladies. A third embodiment of the present invention discloses that the mammalian omentum is processed by first collecting raw mammalian omental material. The mammalian omentum may be either a greater omentum or a lesser omentum of a mammal. The raw mammalian omentum is then cut into smaller pieces. Although in the preferred embodiment, the raw mammalian omental material is initially cut into ¾ inch squares, it should be understood by those skilled in the art that substantial benefit may be derived from using pieces of other sizes and shapes. Then, any blood muscle tissue, kidney tissue, blood veins, arterial vessels, sinew, and membrane tissues are preferably removed.

The raw mammalian omental pieces are then preferably heated into a liquid substance at a temperature of between 100 degrees Fahrenheit and 300 degrees Fahrenheit for a time between 10 minutes and 7.5 hours. According to the preferred embodiment, the omental material should first be heated at a starting temperature of 160 degrees Fahrenheit and the temperature should be raised by 20 degrees Fahrenheit every 2 hours for between 7 and 8 hours. After heating the omental material, it is then filtered. Certain preservatives, vitamins, herbs, and oils having therapeutic qualities and/or specific aromas, such as Vitamin E, lavender, rosemary, comfrey, coconut oil, and almond oil, may be added to the omental liquid substance. The omental liquid substance is then preferably tempered at a temperature of approximately 58 degrees Fahrenheit for approximately 2 hours. Finally, the omentum composition is preferably pressed approximately every 30 minutes. While, in the preferred embodiment, an omentum composition is processed in the above prescribed manner, it should be clearly understood that substantial benefit could be derived from alternative processes in which either alternative steps are present or in which the current steps are used but with temperatures or times that deviate, even substantially, from the preferred times and temperatures.

The inventor has also developed an alternative method of processing mammalian omentum. A fourth embodiment of the present invention describes a method comprising, in combination, the steps of collecting raw mammalian omental material and blending the omental material into a mixture of raw lipid oil. The raw lipid oil mixture is then preferably filtered. Certain herbs, oils, fruits, vegetables, nuts, flowers, plants, trees, roots, leaves, bark, petals, wheat germ, vitamins, minerals, animal fats, ground bone meal/marrow, and water may be added to the composition to enhance the medicinal value of the lipid oil mixture.

Yet another method of processing mammalian omentum has been devised by the inventor. A fifth embodiment of the present invention discloses a method comprising the steps of collecting raw mammalian omental material and blending a first portion of the raw omental material into a mixture of raw lipid oil. A second portion of the raw omental material is then heated into a liquid substance. Finally, the raw lipid oil is combined with the liquid substance and preferably certain herbs, oils, fruits, vegetables, nuts, flowers, plants, trees, roots, leaves, bark, petals, wheat germ, vitamins, minerals, animal fats, ground bone meal/marrow, and water may be added as the liquid substance cools.

While the invention has been particularly claimed, it will be understood by those skilled in the art that the foregoing and other changes in form and details may be made therein without departing from the spirit and scope of the invention. For example, although heating mammalian omentum is one method for extracting lipids from raw omental material, it should be clearly understood that substantial benefit could be derived from other methods of extraction, such as through chemical processes, etc., so long as lipids are extracted from the mammalian omentum. Additionally, there are many types of preservatives, oils, supplements, extracts, minerals and other ingredients which can be combined with the omental lipids in order to alter the scent, the consistency, and the effect of the omentum for treatment of a mammal. Preferably, a blended formula containing approximately 40% aloe, approximately 28% Vitamin E, approximately 28% rose hips, approximately 3% bitter orange, and approximately 1% chamomile will be added to the omentum composition. It should be understood, however, that substantial benefit may be derived from using varying proportions and combinations and that the lists contained herein are merely examples of some of those ingredients.

I claim:

1. A method of treating mammals comprising the step of topically applying to a needed area on said mammal a therapeutically effective amount of a composition comprising a therapeutically effective amount of mammalian omentum.

2. The method of claim 1 wherein said effective amount of said mammalian omentum being between 1% and 100% of said composition.

3. The method of claim 1 wherein said effective amount of said mammalian omentum being approximately 95% of said composition.

4. The composition of claim 1 further comprising mammalian placenta.

5. The method of claim 1 wherein said needed area being an area having a vaginal infection.

6. The method of claim 1 wherein said needed area being an area having hemorrhoids.

7. The method of claim 1 wherein said needed area being the clitoris.

8. The method of claim 1 wherein said needed area being a nipple area of the body.

9. The method of claim 1 wherein said needed area being an eye area of the body.

10. The method of claim 1 wherein said needed area being at least one tendon.

11. The method of claim 1 wherein said needed area being cartilage.

12. The method of claim 1 wherein said needed area being at least one ligament.

13. The method of claim 1 wherein said needed area being a tooth area of a mouth.

14. The method of claim 1 further comprising the step of providing a vaginal cream, said vaginal cream containing a therapeutically effective amount of said mammalian omentum.

15. The method of claim 1 further comprising the step of providing an aftershave, said aftershave containing a therapeutically effective amount of said mammalian omentum.

16. The method of claim 1 further comprising the step of providing a soap, said soap containing a therapeutically effective amount of said mammalian omentum.

17. The method of claim 1 further comprising the step of providing a deodorant, said deodorant containing a therapeutically effective amount of said mammalian omentum.

18. The method of claim 1 further comprising the step of providing a hair gel, said hair gel containing a therapeutically effective amount of said mammalian omentum.

19. The method of claim 1 further comprising the step of providing a skin filler, said skin filler containing a therapeutically effective amount of said mammalian omentum.

20. The method of claim 1 further comprising the step of providing a shampoo, said shampoo containing a therapeutically effective amount of said mammalian omentum.

21. The method of claim 1 further comprising the step of providing a wound ointment, said wound ointment containing a therapeutically effective amount of said mammalian omentum.

22. The method of claim 1 further comprising the step of providing a sun block, said sun block containing a therapeutically effective amount of said mammalian omentum.

23. The method of claim 1 further comprising the step of providing a skin cream, said skin cream containing a therapeutically effective amount of said mammalian omentum.

24. The method of claim 1 further comprising the step of providing an eye cream, said eye cream containing a therapeutically effective amount of said mammalian omentum.

25. The method of claim 1 further comprising the step of providing a lip balm, said lip balm containing a therapeutically effective amount of said mammalian omentum.

26. The method of claim 1 wherein said composition further comprises at least one of herbs, fruits, vegetables, nuts, flowers, plants, trees, roots, leaves, bark, petals, wheat germ, vitamins, minerals, animal fats, ground bone meal/marrow, and water.

27. The method of claim 1 wherein said composition further comprises a therapeutically effective amount of a preservative.

28. The method of claim 27 wherein said preservative being Vitamin E.

29. The method of claim 1 wherein said mammalian omentum being fetal mammalian omentum.

30. The method of claim 29 wherein said fetal mammalian omentum being bovine fetal mammalian omentum.

31. The method of claim 1 wherein said composition further comprises a therapeutically effective amount of Vitamin A.

32. The method of claim 1 wherein said composition further comprises a therapeutically effective amount of Vitamin B.

33. The method of claim 1 wherein said composition further comprises a therapeutically effective amount of Vitamin C.

34. The method of claim 1 wherein said composition further comprises a therapeutically effective amount of Vitamin D.

35. The method of claim 1 wherein said composition further comprises a therapeutically effective amount of a scent.

36. The method of claim 35 wherein said scent being coconut extract.

37. The method of claim 1 wherein said composition further comprises a therapeutically effective amount of mammalian placenta.

38. The method of claim 1 wherein said composition further comprises a therapeutically effective amount of lavender.

39. The method of claim 1 wherein said composition further comprises a therapeutically effective amount of rose hips.

40. The method claim 1 wherein said composition further comprises a therapeutically effective amount of bitter orange.

41. The method of claim 1 wherein said composition further comprises a therapeutically effective amount of chamomile.

42. The method of claim 1 wherein said composition further comprises a therapeutically effective amount of rosemary.

43. The method of claim 1 wherein said composition further comprises a therapeutically effective amount of comfrey.

44. The method of claim 1 wherein said composition further comprises a therapeutically effective amount of allantoin.

45. The method of claim 1 wherein said composition further comprises a therapeutically effective amount of almond oil.

46. The method of claim 1 wherein said composition further comprises a therapeutically effective amount of coconut oil.

47. The method of claim 1 wherein said composition further comprises a therapeutically effective amount of aloe.

48. The method of claim 1 wherein said composition further comprises a therapeutically effective amount of carrier oil.

49. The method of claim 1 wherein said composition further comprises a therapeutically effective amount of essential oil.

50. The method of claim 1 further comprising the step of adding a blended formula to said composition, said blended formula comprising:
a therapeutically effective amount of aloe;
a therapeutically effective amount of Vitamin E;
a therapeutically effective amount of rose hips;
a therapeutically effective amount of bitter orange; and
a therapeutically effective amount of chamomile.

51. The method of claim 50 wherein said therapeutically effective amount of aloe being approximately 40% by volume of said blended formula.

52. The method of claim 50 wherein said therapeutically effective amount of Vitamin E being approximately 28% by volume of said blended formula.

53. The method of claim 50 wherein said therapeutically effective amount of rose hips being approximately 28% by volume of said blended formula.

54. The method of claim 50 wherein said therapeutically effective amount of bitter orange being approximately 3% by volume of said blended formula.

55. The method of claim 50 wherein said therapeutically effective amount of chamomile being approximately 1% by volume of said blended formula.

56. A method for processing mammalian omentum comprising, in combination, the steps of:
collecting raw mammalian omental material; and
heating said raw mammalian omental material into a liquid substance.

57. The method of claim 56 further comprising the step of filtering said liquid substance through a filter.

58. The method of claim 56 further comprising the step of cutting said raw mammalian omental material into pieces of a predetermined size prior to heating said omental material.

59. The method of claim 56 wherein said pieces are approximately ¾ inch squares.

60. The method of claim 56 further comprising the step of removing from said raw mammalian omental material at least one of blood muscle tissue, kidney tissue, blood veins, arterial vessels, sinew, and membrane tissues prior to heating said omental material.

61. The method of claim 56 wherein said raw mammalian omental material comprises the greater omentum of a mammal.

62. The method of claim 56 wherein said raw mammalian omental material comprises the lesser omentum of a mammal.

63. The method of claim 56 further comprising the step of freezing said raw mammalian omental material.

64. The method of claim 56 further comprising the step of tempering said raw mammalian omental material.

65. The method of claim 64 further comprising the step of tempering said raw mammalian omental material at a temperature of approximately 58 degrees Fahrenheit for approximately 2 hours.

66. The method of claim 56 further comprising the step of heating said omental material at a temperature of between 100 degrees Fahrenheit and 300 degrees Fahrenheit for a time between 10 minutes and 7.5 hours.

67. The method of claim 66 further comprising the steps of:
heating said omental material at a starting temperature of 160 degrees Fahrenheit; and
raising the temperature 20 degrees Fahrenheit every 2 hours for between 7 and 8 hours.

68. The method of claim 67 further comprising the step of pressing said omental material.

69. The method of claim 68 further comprising the step of pressing said omental material approximately every 30 minutes.

70. The method of claim 56 further comprising the step of adding a scent to said liquid substance.

71. The method of claim 70 wherein said scent being coconut oil.

72. The method of claim 70 wherein said scent being almond oil.

73. The method of claim 56 further comprising the step of adding a preservative to said liquid substance.

74. The method of claim 73 wherein said preservative being Vitamin E.

75. The method of claim 56 further comprising the step of adding lavender to said liquid substance.

76. The method of claim 56 further comprising the step of adding rosemary to said liquid substance.

77. The method of claim 56 further comprising the step of adding comfrey to said liquid substance.

78. The method of claim 56 further comprising the step of adding a blended formula to said omental material, said blended formula comprising:
a therapeutically effective amount of aloe;
a therapeutically effective amount of Vitamin E;
a therapeutically effective amount of rose hips;
a therapeutically effective amount of bitter orange; and
a therapeutically effective amount of chamomile.

79. The method of claim 78 wherein said therapeutically effective amount of aloe being approximately 40% by volume of said blended formula.

80. The method of claim 78 wherein said therapeutically effective amount of Vitamin E being approximately 28% by volume of said blended formula.

81. The method of claim 78 wherein said therapeutically effective amount of rose hips being approximately 28% by volume of said blended formula.

82. The method of claim 78 wherein said therapeutically effective amount of bitter orange being approximately 3% by volume of said blended formula.

83. The method of claim 78 wherein said therapeutically effective amount of chamomile being approximately 1% by volume of said blended formula.

84. A method of treating mammals comprising the step of ingesting a therapeutically effective amount of a composition comprising a therapeutically effective amount of mammalian omentum.

85. The method of claim 84 wherein said mammalian omentum treats at least one ulcer.

86. A method of processing mammalian omentum comprising, in combination, the steps of:
collecting raw mammalian omental material; and
blending said omental material into a mixture of raw lipid oil.

87. The method of claim 86 further comprising the step of filtering said mixture of raw lipid oil at least once.

88. A method of processing mammalian omentum comprising, in combination, the steps of:
collecting raw mammalian omental material;
blending a first portion of said raw omental material into a mixture of raw lipid oil;
heating a second portion of said raw omental material into a liquid substance; and
combining said raw lipid oil to said liquid substance as said liquid substance cools.
